(19) United States
(12) Patent Application Publication
(10) Pub. No.: US 2023/0181010 A1
(43) Pub. Date: Jun. 15, 2023

(54) PART OF ITEM CONTAINER FOR MEDICAL DEVICE, ITEM CONTAINER FOR MEDICAL DEVICE, MEDICAL DEVICE STORAGE PACK, AND ITEM FOR MEDICAL DEVICE

(71) Applicant: OLYMPUS CORPORATION, Tokyo (JP)

(72) Inventor: Mizuki SUZUKI, Tokyo (JP)

(73) Assignee: OLYMPUS CORPORATION, Tokyo (JP)

(21) Appl. No.: 18/101,192

(22) Filed: Jan. 25, 2023

Related U.S. Application Data
(63) Continuation of application No. PCT/JP2020/029988, filed on Aug. 5, 2020.

(51) Int. Cl. A61B 1/00; A61B 50/20

(52) U.S. Cl. CPC A61B 1/00147; A61B 50/20

(57) ABSTRACT

A part of an item container for a medical device includes a bulging-shape portion for storing an item for a medical device, a flange, a protruding-shape portion, and a concave portion provided in contact with the bulging-shape portion. The protruding-shape portion is arranged at a position spaced apart from the bulging-shape portion in a second direction, and is provided to extend in an elongated manner in a third direction different from the second direction.

### CROSS REFERENCE TO RELATED APPLICATION

This application is a continuation application of PCT/JP2020/029988 filed on Aug. 5, 2020, the entire contents of which are incorporated herein by this reference.

### BACKGROUND OF THE INVENTION

### 1. Field of the Invention

The present invention relates to a part of an item container for a medical device for storing an item for a medical device, an item container for a medical device, a medical device storage pack, and an item for a medical device.

### 2. Description of the Related Art

Containers for storing items have been widely used in various fields. As for a container for storing an item for a medical device, in particular, the interior of the container is maintained in a sterile condition.

For example, Japanese Patent Application Laid-Open Publication No. 2000-225126 discloses a medical kit container in which a sterile paper is adhered to a flange portion provided around a storage portion to seal the storage portion. Here, the flange portion is formed with a convex portion for more reliable sealing.

A medical kit is stored in the storage portion of the medical kit container, the sterile paper is adhered to the flange portion to seal the storage portion of the container, and the container is irradiated with gamma rays or electron beams to perform ethylene oxide treatment to produce a sterilized medical kit pack.

### SUMMARY OF THE INVENTION

A part of an item container for a medical device according to an aspect of the present invention includes: a bulging-shape portion configured to bulge in a first direction and to store an item for a medical device in a hollow interior; a flange provided at a peripheral edge of the bulging-shape portion; a protruding-shape portion protruding from the flange in the first direction; and a concave portion provided in contact with the bulging-shape portion, wherein the protruding-shape portion is arranged at a position spaced apart from the bulging-shape portion in a second direction and is provided to extend in an elongated manner in a third direction different from the first direction and the second direction.

An item container for a medical device according to an aspect of the present invention includes: a part of an item container for a medical device including: a bulging-shape portion configured to bulge in a first direction and to store an item for a medical device in a hollow interior; a flange provided at a peripheral edge of the bulging-shape portion; a protruding-shape portion protruding from the flange in the first direction; and a concave portion provided in contact with the bulging-shape portion, wherein the protruding-shape portion is arranged at a position spaced apart from the bulging-shape portion in a second direction and is provided to extend in an elongated manner in a third direction different from the first direction and the second direction; and a seal body bonded to the part of the item container for the medical device.

A medical device storage pack according to an aspect of the present invention, includes: a part of an item container for a medical device including: a bulging-shape portion configured to bulge in a first direction and to store an item for a medical device in a hollow interior; a flange provided at a peripheral edge of the bulging-shape portion; a protruding-shape portion protruding from the flange in the first direction; and a concave portion provided in contact with the bulging-shape portion, wherein the protruding-shape portion is arranged at a position spaced apart from the bulging-shape portion in a second direction and is provided to extend in an elongated manner in a third direction different from the first direction and the second direction; the item for the medical device stored in the part of the item container for the medical device; and a seal body bonded to the part of the item container for the medical device to seal a periphery of the item for the medical device.

An item for a medical device according to an aspect of the present invention is configured to be stored in an item container for a medical device including: a part of an item container for a medical device including: a bulging-shape portion configured to bulge in a first direction and to store an item for a medical device in a hollow interior; a flange provided at a peripheral edge of the bulging-shape portion; a protruding-shape portion protruding from the flange in the first direction; and a concave portion provided in contact with the bulging-shape portion, in which the protruding-shape portion is arranged at a position spaced apart from the bulging-shape portion in a second direction and is provided to extend in an elongated manner in a third direction different from the first direction and the second direction; and a seal body bonded to the part of the item container for the medical device, wherein the item for the medical device is stored in the hollow interior of the bulging-shape portion.

### BRIEF DESCRIPTION OF THE DRAWINGS

FIG. 1 is a plan view illustrating an item container for a medical device in a first embodiment of the present invention.

FIG. 2 is a cross-sectional view taken along the line F2-F2 of FIG. 1, illustrating the item container for a medical device in the above first embodiment.

FIG. 3 is a view for explaining an operation when a medical device storage pack storing an item for a medical device in the item container is held by a hand and unsealed in the above first embodiment.

FIG. 4 is a view illustrating a position of an item for a medical device which can be taken out according to an unsealing state of a seal body in the above first embodiment.

FIG. 5 is a perspective view illustrating a state in which a plurality of medical device storage packs is stored in two rows in a box in the above first embodiment.

FIG. 6 is a plan view illustrating a state in which an even number of medical device storage packs are stored in two rows in the box in the above first embodiment.

FIG. 7 is an enlarged view of a main part illustrating a state in which an outer surface of a spherical concave portion and an outer surface of a protruding portion of the respective medical device storage packs stored in two rows come into contact with each other to suppress the movement of the medical device storage packs in the box in the above first embodiment.

FIG. 8 is a plan view illustrating a state in which an odd number of medical device storage packs are stored in two rows in the box in the above first embodiment.

### DETAILED DESCRIPTION OF THE PREFERRED EMBODIMENT(S)

Hereinafter, an embodiment of the present invention will be described with reference to the accompanying drawings. However, the present invention is not limited to the embodiment described below.

Note that in the description of the drawings, the same or corresponding elements are denoted by the same reference numerals as appropriate. In addition, it should be noted that the drawings are schematic, and a relationship between lengths of elements, a ratio of lengths of elements, and the like in one drawing may be different from actual ones. Further, a plurality of drawings may include portions having different relationships or ratios of lengths.

### First Embodiment

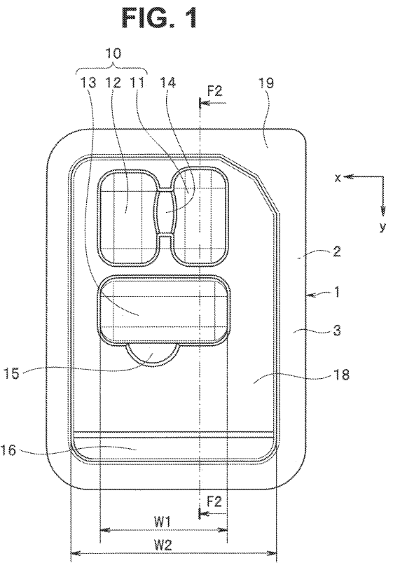

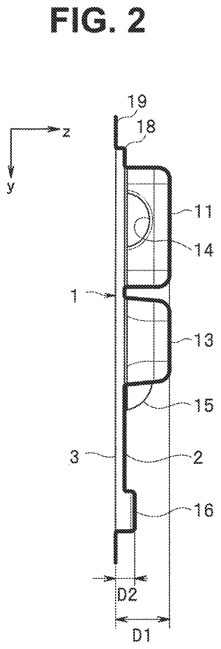

FIG. 1 to FIG. 8 illustrate a first embodiment of the present invention, in which FIG. 1 is a plan view illustrating an item container 1 for a medical device, and FIG. 2 is a cross-sectional view taken along the line F2-F2 of FIG. 1 illustrating the item container 1 for a medical device.

The item container 1 is a container for storing an item for a medical device in a sterile condition, and is configured as, for example, a blister pack. Here, the item for a medical device is a medical device, a replacement part of a medical device, an accessory of a medical device, or the like, and specific examples thereof will be described later.

The item container 1 for a medical device includes a main body 2 as a first part and a seal body 3 as a second part.

The main body 2 includes a bulging portion 10 (bulging-shape portion), a protruding portion 16 (protruding-shape portion), and a substantially planar flange portion 19 (flange) that connects the bulging portion 10 and the protruding portion 16. Here, for ease of explanation, x, y, and z directions are set as three-dimensional orthogonal directions. As illustrated in FIG. 1 and FIG. 2, etc., the x direction is a short-side direction of the item container 1, the y direction is a long-side direction of the item container 1, and the z direction is a thickness direction of the item container 1.

The bulging portion 10 bulges from the flange portion 19 in the z direction (first direction) and is configured to store an item for a medical device in a hollow interior (storage portion as a concave portion).

The bulging portion 10 of the present embodiment includes a plurality of bulging portions, specifically, three bulging portions 11, 12, and 13 (bulging-shape portions), and can store three items for the medical device. However, the number of items stored in the item container 1 may be any number as long as it is one or more. Therefore, the number of bulging portions may be an arbitrary number according to the number of items.

For example, a cylindrical concave portion 14 (a concave portion having a curved surface) forming a semi-cylindrical surface is provided between the bulging portion 11 and the bulging portion 12. The cylindrical concave portion 14 connects the hollow inside the bulging portion 11 and the hollow inside the bulging portion 12. The cylindrical concave portion 14 facilitates pinching of the items each stored in the bulging portions 11 and 12 with fingers.

In addition, for example, a spherical concave portion 15 (a concave portion having a curved surface) forming about half of a hemispherical surface is provided on the protruding portion 16 side of the bulging portion 13. The spherical concave portion 15 facilitates pinching of the item stored in the bulging portion 13 with fingers.

The flange portion 19 is a substantially planar portion provided on a peripheral edge of the bulging portion 10. The flange portion 19 is provided with a stepped concave portion 18 which is a portion not bonded to the seal body 3.

The protruding portion 16 is disposed at a position spaced apart from the bulging portion 10 in the y direction (second direction) and protrudes from the flange portion 19 in the z direction (first direction). The y direction (second direction) in which the protruding portion 16 is separated from the bulging portion 10 is orthogonal to the arrangement direction of at least two bulging portions among the plurality of bulging portions 11, 12, and 13, here, the arrangement direction (x direction) of the bulging portion 11 and the bulging portion 12.

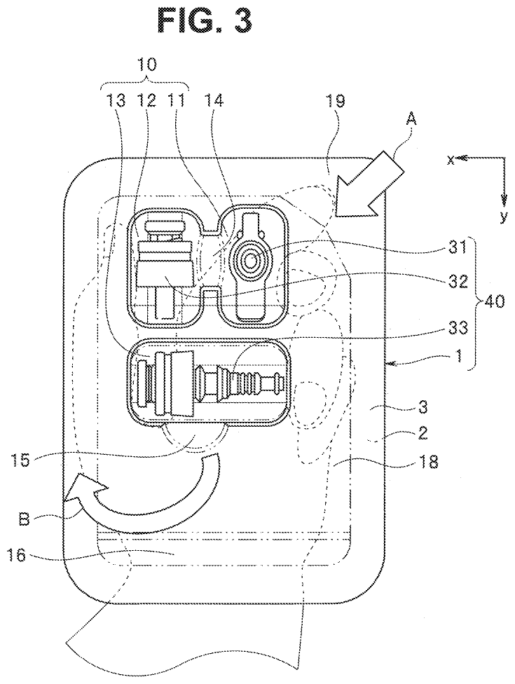

Here, a distance by which the protruding portion 16 is separated from the bulging portion 10 is such that the base of the thumb or a part of the palm of the hand of a user holding the bulging portion 10 of the item container 1 can enter between the bulging portion 10 and the protruding portion 16 (see FIG. 3).

Specifically, similar to the bulging portion 10 described above, the protruding portion 16 is provided as another bulging portion (another bulging-shape portion) that bulges in the z direction (first direction) and has a hollow interior.

Note that since there is no need to store an item inside the protruding portion 16, it is not prohibited that the inside of the protruding portion 16 is configured to be dense (that is, in a state to be filled with a material forming the main body 2). However, by configuring the protruding portion 16 as another bulging portion having a hollow interior, the protruding portion 16 can be manufactured in the same process as the bulging portion 10, and the weight of the item container 1 can be reduced. Therefore, the protruding portion 16 has nothing inside except gas (air, gas for sterilization, etc.).

The protruding portion 16 is provided to extend in an elongated manner in the x direction (third direction along the flange portion 19) different from the z direction (first direction) and the y direction (second direction). Note that, here, the x direction orthogonal to the y direction is given as a preferable example of the third direction in which the protruding portion 16 extends in an elongated manner, but the third direction is not limited thereto, and the third direction in which the protruding portion 16 extends in an elongated manner may be a direction different from the y direction along an xy plane.

As illustrated in FIG. 1, a length W2 of the protruding portion 16 in the x direction (third direction) is preferably larger than a length W1 of the bulging portion 10 in the x direction (third direction). The length W2 is preferably larger than the length W1 because, as will be described later with reference to FIG. 3, when a hand holding a medical device storage pack 40 that stores the item for a medical device (for example, a forceps plug 31, a suction button 32, and an air/water supply button 33 as illustrated in FIG. 3) in the item container 1 comes into contact with the protruding portion 16, the item container 1 is not displaced even when a force for unsealing the medical device storage pack 40 is applied, and the seal body 3 is reliably and easily unsealed.

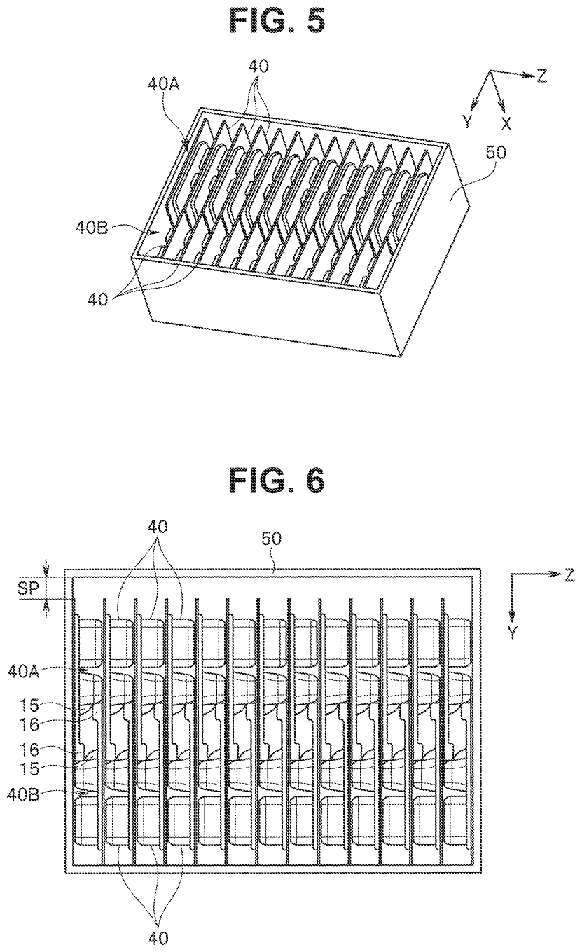

Furthermore, as illustrated in FIG. 2, a length D2 of the protruding portion 16 in the z direction (first direction) is preferably smaller than a length D1 of the bulging portion 10 in the z direction (first direction). The length D2 is preferably smaller than the length D1 because, as will be described later with reference to FIG. 5 to FIG. 8, when a plurality of medical device storage packs 40 is stored in a box 50, a storage property (use efficiency of the space in the box 50 at the time of storage) is enhanced and the movement of the medical device storage packs 40 in the box 50 is suppressed.

The seal body 3 is bonded to the flange portion 19 so as to cover at least an opening of the bulging portion 10 (seal the opening while maintaining a sterile condition). Specifically, the seal body 3 of the present embodiment has a substantially rectangular shape having substantially the same side edge as the side edge of the flange portion 19 of the main body 2, and is configured to cover not only the opening of the bulging portion 10 but also an opening of the protruding portion 16 (another bulging portion).

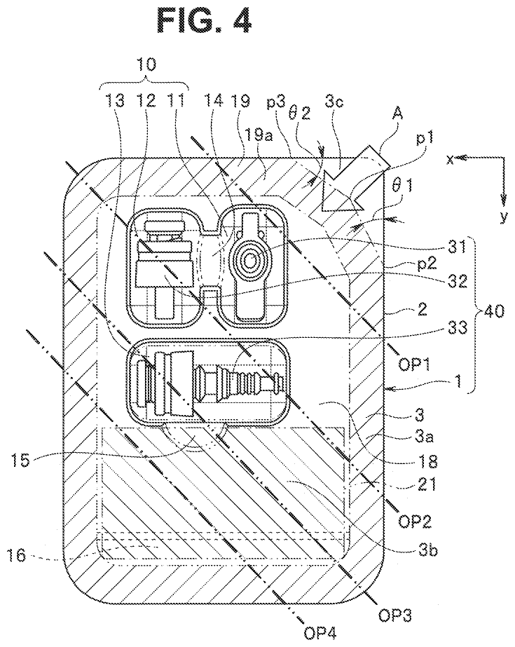

As illustrated in a portion hatched from the upper right to the lower left in FIG. 4, the seal body 3 is bonded to a bonding surface 19a of the flange portion 19 on an outer peripheral side of the stepped concave portion 18 in the main body 2 at the bonding portion 3a of the peripheral edge. At the time of the bonding, a tab portion 3c (non-bonding portion, non-bonding surface) which is not bonded to the flange portion 19 is provided in an upper right of the seal body 3 (position spaced from the bulging portion 10). Since the tab portion 3c is not bonded to the flange portion 19, the tab portion 3c can be easily pinched by fingers.

Note that here, since it is assumed that the medical device storage pack 40 is held by the left hand and the seal body 3 is unsealed by the right hand, the tab portion 3c is provided in an upper right portion of FIG. 4, but the present invention is not limited to have the tab portion 3c provided in the upper right portion of FIG. 4.

For example, the tab portion 3c may be provided in an upper left portion of FIG. 4 for the use of holding the medical device storage pack 40 by the right hand and unsealing the seal body 3 by the left hand.

Alternatively, the tab portion 3c may be provided in both the upper right portion and the upper left portion in FIG. 4 to allow the medical device storage pack 40 to be held by either the right hand or the left hand and allow the seal body 3 to be unsealed by either the left hand or the right hand.

As illustrated in FIG. 4, a boundary between the bonding portion 3a and the tab portion 3c forms a broken line having a central apex p1, an apex p2 at a position intersecting with a right side of the seal body 3, and an apex p3 at a position intersecting with an upper side of the seal body 3, and an angle formed by the boundary of the bonding portion 3a at each of the apexes p1, p2, and p3 is an obtuse angle.

To be specific, an angle θ1 formed by the right side of the seal body 3 and a line segment p2p1 is, for example, 30°, an angle θ2 formed by the upper side of the seal body 3 and a line segment p3p1 is, for example, 30°, and the angle of the boundary of the bonding portion 3a at each of the apexes p1, p2, and p3 is, for example, 150°.

Note that in the example illustrated in FIG. 4, since the bonding portion 3a has a constant width, the boundary on an inner peripheral side of the bonding portion 3a in the vicinity of the tab portion 3c has a shape similar to the boundary on an outer peripheral side of the bonding portion 3a, that is, a shape having three apexes each forming the obtuse angle. However, since it is not essential for the bonding portion 3a to have a constant width, the boundary on the inner peripheral side of the bonding portion 3a may have, for example, an arc shape or another shape.

In addition, although the number of apexes at the boundary between the bonding portion 3a and the tab portion 3c is three here, the number may be four or more.

At least one of the main body 2 and the seal body 3 described above, and preferably both of them are made of a material having optical transparency (for example, clear or translucent) so that an item for a medical device stored inside can be visually checked.

Specifically, an example of the material constituting the main body 2 is a laminated resin in which polyethylene terephthalate (PET) and a PET-based resin are laminated. In addition, an example of a material constituting the seal body 3 is a laminated resin in which polyethylene (PE) and a PE-based resin are laminated.

By using a combination of these materials for the main body 2 and the seal body 3, the main body 2 and the seal body 3 can be bonded by thermal bonding or thermal welding, and a bonding step using an adhesive or the like is not required. Furthermore, by using a laminated resin for the main body 2 and the seal body 3, a sealing property (watertightness and airtightness) of the bonding portion can be maintained, and a sterilization property of the stored item can be favorably maintained.

However, the combination of the materials constituting the main body 2 and the seal body 3 is not limited to the above-described example, and appropriate materials may be used as long as the main body 2 and the seal body 3 can be bonded by thermal bonding or thermal welding and the sealing property and the sterilization property can be maintained. In the way described above, the seal body 3 is bonded to the flange portion 19 in a state where an item for a medical device is stored in the item container 1, completing the medical device storage pack 40 in which a periphery of the item for the medical device is sealed.

Meanwhile, the medical device storage pack 40 in which the item for the medical device is stored in the item container 1 becomes a sterilized pack by applying sterilization treatment. Such sterilization treatment includes radiation sterilization, gamma radiation sterilization, gas sterilization (e.g., ethylene oxide gas (EOG) sterilization), and the like.

When radiation sterilization or gamma radiation sterilization is performed as the sterilization treatment, the above-described laminated resin can be used as the material of the main body 2 and the seal body 3. Meanwhile, when gas sterilization is performed, it is necessary to form the seal body 3 by using a porous material such as sterile paper having air permeability. When the gas sterilization is performed, the seal body 3 made of, for example, sterile paper is bonded to the main body 2 using an adhesive or the like.

On the seal body 3 of the sterilized medical device storage pack 40, a traceability seal 21 for tracing the process of production and distribution is attached. On the traceability seal 21, for example, a two-dimensional bar code, a manufacturer name, and the like are written.

For example, as indicated by a portion 3b hatched from the upper left to the lower right in FIG. 4, the traceability seal 21 is attached to a position on the stepped concave portion 18 and the protruding portion 16 on the y-direction side with respect to the bulging portion 10. As described above, the traceability seal 21 is attached to a position not overlapping an item for a medical device so that the item for the medical device stored inside can be seen.

Accordingly, it is possible to easily check whether or not the item for the medical device is reliably stored in the medical device storage pack 40 by, for example, a camera (or visual observation by a human) at the time of manufacturing. Further, the traceability seal 21 can be easily checked by a camera (or visual observation by a human) at the same time as checking of the stored item for the medical device.

Note that in the example of FIG. 4, the traceability seal 21 is attached so that the entirety of all the items for the medical device can be seen when viewed in the z direction, but the present invention is not limited thereto. For example, the traceability seal 21 may be attached so that at least a part of all the stored items for the medical device can be seen. The above is because it is possible to confirm that the target item is stored when at least a part of the item is visible.

Next, FIG. 3 is a view for explaining the operation when the medical device storage pack 40 storing an item for a medical device in the item container 1 is held by a hand and unsealed, and FIG. 4 is a view illustrating a position of an item for a medical device which can be taken out according to the unsealed state of the seal body 3. Note that in FIG. 3 and FIG. 4, the corresponding portion is illustrated in a see-through manner so that the item for the medical device stored in the bulging portion 10 can be seen.

In the present embodiment, the forceps plug 31 is stored in the bulging portion 11, the suction button 32 is stored in the bulging portion 12, and the air/water supply button 33 is stored in the bulging portion 13. The forceps plug 31, the suction button 32, and the air/water supply button 33 of the digestive system endoscope are examples of the items for the medical device, which are often sold together in one package.

Note that although the digestive system endoscope has been described here as an example of the medical device, the endoscope is not limited to the digestive system, and may be endoscopes in other fields. Furthermore, the item for the medical device is not limited to an item for an endoscope, but may be, for example, an item for a treatment instrument such as an energy treatment instrument, or an item for another device used for medical treatment.

When the medical device storage pack 40 is unsealed, the bulging portion 10 is held so as to be sandwiched between the thumb of one hand, the left hand in the example of FIG. 3, and the fingers other than the thumb. When the bulging portion 10 is held, the base of the thumb or a part of the palm of the left hand enters between the bulging portion 10 and the protruding portion 16 and comes into contact with the protruding portion 16.

In the state above, the tab portion 3c is pinched by the fingers (for example, the thumb and the index finger) of the right hand and moved in a direction of an arrow A in FIG. 3 to start unsealing of the seal body 3. At the time of unsealing the seal body 3, although the medical device storage pack 40 is likely to rotate, for example, in a direction of an arrow B in FIG. 3, the rotation can be prevented because the base of the thumb or a part of the palm of the left hand is in contact with the protruding portion 16.

Accordingly, the force in the direction of the arrow A for unsealing the seal body 3 is not used for the positional deviation of the medical device storage pack 40 but is effectively used for peeling the bonding portion 3a.

Further, at the time of the peeling of the bonding portion 3a, since not only the apexes p2 and p3 on both sides but also the central apex p1 is further provided at the boundary between the bonding portion 3a and the tab portion 3c, for example, the peeling of the bonding portion 3a can be started from the apex p1 by only pulling the tab portion 3c naturally, without tilting and pulling the tab portion 3c so as to start the peeling of the bonding portion 3a from the apex p2 or the apex p3. In the way described above, it is possible to increase the probability that the peeling of the bonding portion 3a starts from any of the apexes p1, p2, and p3.

As a result, when the tab portion 3c is pinched and moved in the direction of the arrow A, the unsealing force in the direction of the arrow A first acts in a concentrated manner on any one of the apexes p1, p2, and p3 (for example, the apex p1), and the peeling of the bonding portion 3a can be easily started without requiring a large force.

As just described, once the peeling is started from any one of the apexes p1, p2, and p3 as a starting point, the peeling of the bonding portion 3a proceeds smoothly by continuing to apply an appropriate force thereafter.

At the time when the peeling of the bonding portion 3a reaches an unsealing position OP1 in FIG. 4, none of the stored forceps plug 31, the suction button 32, and the air/water supply button 33 can be taken out yet.

When the peeling of the bonding portion 3a reaches an unsealing position OP2 of FIG. 4, the forceps plug 31 can be taken out. In the state above, by inserting fingers into the cylindrical concave portion 14, the forceps plug 31 can be easily pinched and taken out. Additionally, in the state above, the suction button 32 and the air/water supply button 33 are covered with the seal body 3 and cannot be taken out. Since the suction button 32 and the air/water supply button 33 are covered with the seal body 3, even when the medical device storage pack 40 is tilted at the time of pinching the forceps plug 31, it is possible to prevent the suction button 32 and the air/water supply button 33 from accidentally falling off.

Subsequently, when the peeling of the bonding portion 3a reaches an unsealing position OP3 in FIG. 4, the suction button 32 can be taken out. In the state above, also, the suction button 32 can be easily pinched and taken out by inserting fingers into the cylindrical concave portion 14. In the state above, the air/water supply button 33 is covered with the seal body 3 and cannot be taken out. Since the air/water supply button 33 is covered with the seal body 3, even when the medical device storage pack 40 is tilted at the time of pinching the suction button 32, it is possible to prevent the air/water supply button 33 from accidentally falling off.

Thereafter, when the peeling of the bonding portion 3a reaches an unsealing position OP4 in FIG. 4, the air/water supply button 33 can be taken out. In the state above, by inserting fingers into the spherical concave portion 15, the air/water supply button 33 can be easily pinched and taken out.

As illustrated in FIG. 1, the bulging portions 11, 12, and 13 are arranged in an upper right portion, an upper left portion, and a center portion of the main body 2, respectively, the forceps plug 31, the suction button 32, and the air/water supply button 33 are stored in the bulging portions 11, 12, and 13 as illustrated in FIG. 3 and FIG. 4, and the tab portion 3c is arranged in the upper right of the seal body 3 in FIG. 4. Therefore, the seal body 3 is unsealed toward the lower left diagonally in FIG. 4, and the forceps plug 31, the suction button 32, and the air/water supply button 33 can be sequentially taken out one by one while preventing from falling off.

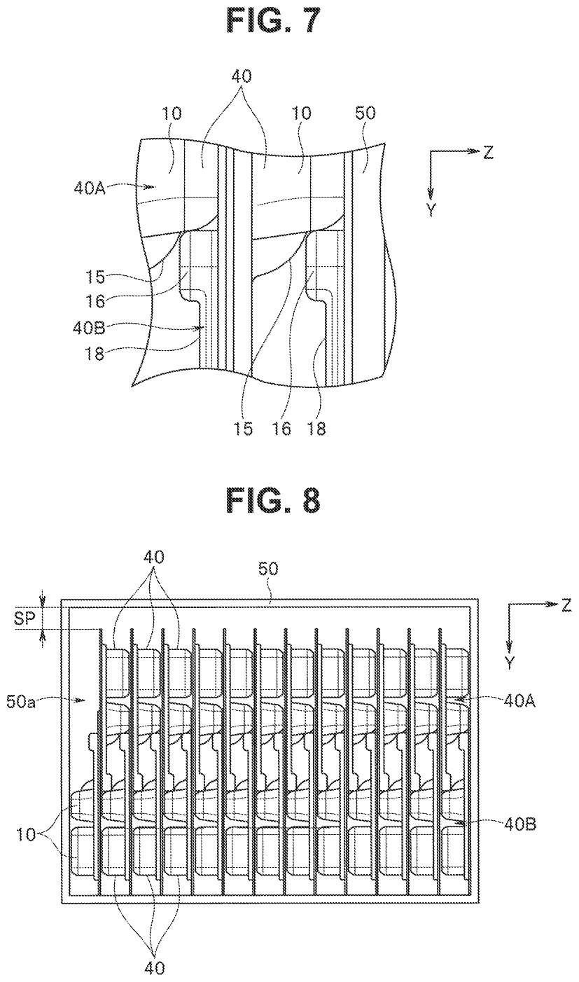

Next, FIG. 5 is a perspective view illustrating a state in which the plurality of medical device storage packs 40 is stored in two rows in the box 50, FIG. 6 is a plan view illustrating a state in which an even number of the medical device storage packs 40 are stored in two rows in the box 50, and FIG. 7 is an enlarged view of the main part illustrating a state in which an outer surface of the spherical concave portion 15 and an outer surface of the protruding portion 16 of the respective medical device storage packs 40 stored in two rows come into contact with each other to suppress the movement of the medical device storage packs 40 in the box 50.

Here, in FIG. 5 to FIG. 7 (and FIG. 8 described later), X, Y, and Z directions are set as three-dimensional orthogonal directions. The X direction is a depth direction of the box 50, the Y direction is a short-side direction of the box 50, and the Z direction is a long-side direction of the box 50. The X, Y, and Z directions are respectively set to be the same as the x, y, and z directions of the medical device storage packs 40 (an upper pack row 40A to be described later) stored on an upper side among the plurality of medical device storage packs 40 stored in two rows in the box 50.

As illustrated in FIG. 5, FIG. 6, etc., the plurality of medical device storage packs 40 is shipped in a state of being stored in the box 50.

FIG. 6 illustrates an example in which an even number (26 in the illustrated example) of the medical device storage packs 40 are stored in two rows in the box 50. Hereinafter, in FIG. 6, the row of the medical device storage packs 40 stored on the upper side is referred to as the upper pack row 40A, and the row of the medical device storage packs 40 stored on a lower side is referred to as a lower pack row 40B.

The medical device storage packs 40 in the lower pack row 40B are stored in the box 50 in a state of being rotated by 180° around an axis in the X direction (that is, facing the opposite side in the Y direction) with respect to the medical device storage packs 40 in the upper pack row 40A.

In the storage state above, the upper pack row 40A and the lower pack row 40B are stored so as to partially overlap in positions in the Y direction. To be specific, the position in the Y direction on the side of the protruding portion 16 including the spherical concave portion 15 of the upper pack row 40A and the position in the Y direction on the side of the protruding portion 16 including the spherical concave portion 15 of the lower pack row 40B overlap each other.

In the portion where the positions in the Y direction overlap, as illustrated in FIG. 7, an outer surface in the −Z direction of the protruding portion 16 of the medical device storage pack 40 in the lower pack row 40B is in contact with an outer surface of the bulging portion 10 of the medical device storage pack 40 in the upper pack row 40A and closely faces an outer surface in the +Z direction of the spherical concave portion 15 of the medical device storage pack 40 in the upper pack row 40A.

Similarly, as illustrated in FIG. 6, an outer surface in the +Z direction of the protruding portion 16 of the medical device storage pack 40 in the upper pack row 40A is in contact with the outer surface of the bulging portion 10 of the medical device storage pack 40 in the lower pack row 40B and closely faces an outer surface in the −Z direction of the spherical concave portion 15 of the medical device storage pack 40 in the lower pack row 40B.

As a result, the plurality of medical device storage packs 40 is spread in the Z direction (+Z direction and −Z direction) as a whole and comes into contact with an inner wall of the box 50, thereby suppressing the movement (not only the movement in the Z direction but also the movement in the Y direction) in the box 50. Therefore, when the box 50 is transported, the medical device storage packs 40 can be prevented from rattling inside the box 50.

Note that in order to enable such a storing method, it is preferable that the total length of the thickness of the spherical concave portion 15 in the z direction and the thickness of the protruding portion 16 in the z direction be substantially the same as the thickness of the bulging portion 10 in the z direction.

In addition, the length of the box 50 in the Y direction is configured such that a space having a length SP (the length SP in the Y direction) is generated inside the box 50 after the medical device storage packs 40 are stored in two rows. By adopting such a configuration, the medical device storage pack 40 can be easily taken out from the box 50 by inserting a finger or the like into the space having the length SP.

FIG. 8 is a plan view illustrating a state in which an odd number of the medical device storage packs 40 are stored in two rows in the box 50. FIG. 8 illustrates a modification of the storage method illustrated in FIG. 6.

In other words, while the number of medical device storage packs 40 stored in the box 50 is an even number in FIG. 6, the number thereof is an odd number (25 in the illustrated example) which is one less than that in the example of FIG. 6 in FIG. 8.

As compared with the example of FIG. 6, in the example of FIG. 8, the medical device storage pack 40 on the leftmost side of the upper pack row 40A is removed to form a space 50a. Note that as in the example of FIG. 6, the space having the length SP in the Y direction is generated inside the box 50.

Accordingly, a hand or a finger can be inserted into the space 50a, and the medical device storage pack 40 can be taken out more easily than the example of FIG. 6.

Also in the storage method above, as in the storage method of FIG. 6, the plurality of medical device storage packs 40 is spread in the Z direction as a whole and comes into contact with the inner wall of the box 50. As a result, for example, the bulging portion 10 of the medical device storage pack 40 on the leftmost side of the lower pack row 40B comes into contact with an inner left wall of the box 50.

However, in the storage method of FIG. 8, the suppression of the movement of the upper pack row 40A in the Z direction is slightly weaker than that in the storage method of FIG. 6, and as a result, the suppression of the movement of the plurality of medical device storage packs 40 as a whole in the Y direction is also slightly weaker. However, even when the storage method of FIG. 8 is adopted, the movement of the medical device storage pack 40 in the Z direction and the Y direction in the box 50 can be suppressed according as the storage method of FIG. 6, and when the box 50 is transported, the rattling of the medical device storage packs 40 in the box 50 can be prevented to some extent.

Note that in the above-described embodiment, the plurality of items for the medical device is stored in the item container 1, but the present invention is not limited to the plurality of items, and one or more items for the medical device may be stored.

Although FIG. 5 to FIG. 8 illustrate the example in which the medical device storage packs 40 are stored in two rows in the box 50, the present invention is not limited thereto, and the medical device storage packs 40 may be stored in one row, or may be stored in a plurality of rows such as three rows or four rows.

Therefore, according to the first embodiment, since the protruding portion 16 is arranged at the position spaced apart from the bulging portion 10 in the second direction and the protruding portion 16 is provided so as to extend in an elongated manner in the third direction different from the second direction (and the first direction), the base of the thumb or a part of the palm of the hand holding the bulging portion 10 of the item container 1 enters between the bulging portion 10 and the protruding portion 16 and comes into contact with the protruding portion 16, thereby preventing rotation or positional displacement of the medical device storage pack 40 and making it possible to easily and reliably unseal the seal body 3.

Then, since the seal body 3 is smoothly unsealed, it is possible to prevent the item for the medical device stored in the bulging portion 10 from accidentally falling off when the medical device storage pack 40 is unsealed.

Further, by forming the protruding portion 16 as another bulging portion having a hollow interior, the protruding portion 16 can be manufactured in the same process as the bulging portion 10, and the weight of the item container 1 can be reduced.

Since the seal body 3 covers not only the opening of the bulging portion 10 but also the opening of the protruding portion 16, the area of the seal body 3 covering the part other than the bulging portion 10 is increased, and the traceability seal 21 can be attached without covering the stored item for the medical device.

In addition, by configuring the third direction to be orthogonal to the second direction, an outer shape of the item container 1 can be reduced while ensuring the distance between the bulging portion 10 and the protruding portion 16.

By configuring the bulging portion 10 to include the plurality of bulging portions 11, 12, and 13, a plurality of items for the medical device can be collectively stored. Thus, the item container 1 suitable for items to be sold as a set is obtained.

Since the second direction is configured to be orthogonal to the arrangement direction of at least the two bulging portions 11 and 12 among the plurality of bulging portions 11, 12, and 13, it is possible to take out the plurality of items for the medical device one by one in order when the tab portion 3c is moved in an oblique direction, as illustrated in FIG. 4.

By making the length D2 of the protruding portion 16 in the first direction smaller than the length D1 of the bulging portion 10 in the first direction, when the medical device storage packs 40 are stored in, for example, two rows in the box, the portions of the upper pack row 40A and the lower pack row 40B other than the bulging portion 10 can be overlapped each other, so that the storage property can be enhanced and the movement of the medical device storage packs 40 in the box 50 can be suppressed.

By configuring the length W2 of the protruding portion 16 in the third direction to be larger than the length W1 of the bulging portion 10 in the third direction, the base of the thumb or a part of the palm of the hand holding the bulging portion 10 can reliably enter between the bulging portion 10 and the protruding portion 16. As a result, the medical device storage pack 40 can be more reliably held at the time of unsealing.

It should be noted that the present invention is not limited to the above-described embodiment as it is, and can be embodied by modifying constituent elements without departing from the gist of the present invention at an implementation stage. In addition, various aspects of the invention can be formed by appropriately combining the plurality of constituent elements disclosed in the above embodiment. For example, some constituent elements may be deleted from all the constituent elements shown in the embodiment. Furthermore, constituent elements in different embodiments may be appropriately combined. As described above, it goes without saying that various modifications and applications can be made without departing from the gist of the invention.

What is claimed is:

1. A part of an item container for a medical device comprising:
a bulging-shape portion configured to bulge in a first direction and to store an item for a medical device in a hollow interior;
a flange provided at a peripheral edge of the bulging-shape portion;
a protruding-shape portion protruding from the flange in the first direction; and
a concave portion provided in contact with the bulging-shape portion,
wherein the protruding-shape portion is arranged at a position spaced apart from the bulging-shape portion in a second direction and is provided to extend in an elongated manner in a third direction different from the first direction and the second direction.

2. The part of an item container for a medical device according to claim 1,
wherein the concave portion has a curved surface.

3. The part of an item container for a medical device according to claim 1,
wherein the flange is provided with a bonding surface to which a seal body is bonded so as to cover at least an opening of the bulging-shape portion.

4. The part of an item container for a medical device according to claim 3,
wherein the protruding-shape portion is another bulging-shape portion bulging in the first direction and having a hollow interior.

5. The part of an item container for a medical device according to claim 1,
wherein the protruding-shape portion is separated from the bulging-shape portion by a distance, and the distance allows a base of a thumb or a part of a palm of a user to enter.

6. The part of an item container for a medical device according to claim 4,
wherein the bonding surface is provided with an opening of the other bulging-shape portion further covered by the seal body.

7. The part of an item container for a medical device according to claim 1,
wherein the third direction is orthogonal to the second direction.

8. The part of an item container for a medical device according to claim 1,
wherein the bulging-shape portion includes a plurality of bulging-shape portions.

9. The part of an item container for a medical device according to claim 8,
wherein the second direction is orthogonal to an arrangement direction of at least two bulging-shape portions of the plurality of bulging-shape portions.

10. The part of an item container for a medical device according to claim 1,
wherein a length of the protruding-shape portion in the first direction is smaller than a length of the bulging-shape portion in the first direction.

11. The part of an item container for a medical device according to claim 1,
wherein a length of the protruding-shape portion in the third direction is larger than a length of the bulging-shape portion in the third direction.

12. The part of an item container for a medical device according to claim 1,
wherein the protruding-shape portion has nothing inside except for gas.

13. The part of an item container for a medical device according to claim 3,
wherein a non-bonding surface between the flange and the seal body is provided at a position spaced apart from the bulging-shape portion, and
a boundary between the non-bonding surface and the bonding surface has at least three apexes.

14. An item container for a medical device comprising:
a part of an item container for a medical device including:
a bulging-shape portion configured to bulge in a first direction and to store an item for a medical device in a hollow interior;
a flange provided at a peripheral edge of the bulging-shape portion;
a protruding-shape portion protruding from the flange in the first direction; and
a concave portion provided in contact with the bulging-shape portion,
wherein the protruding-shape portion is arranged at a position spaced apart from the bulging-shape portion in a second direction and is provided to extend in an elongated manner in a third direction different from the first direction and the second direction; and
a seal body bonded to the part of the item container for the medical device.

15. A medical device storage pack comprising:
a part of an item container for a medical device including:
a bulging-shape portion configured to bulge in a first direction and to store an item for a medical device in a hollow interior;
a flange provided at a peripheral edge of the bulging-shape portion;
a protruding-shape portion protruding from the flange in the first direction; and
a concave portion provided in contact with the bulging-shape portion,
wherein the protruding-shape portion is arranged at a position spaced apart from the bulging-shape portion in a second direction and is provided to extend in an elongated manner in a third direction different from the first direction and the second direction;
the item for the medical device stored in the part of the item container for the medical device; and
a seal body bonded to the part of the item container for the medical device to seal a periphery of the item for the medical device.

16. The medical device storage pack according to claim 15,
wherein the item for the medical device is a forceps plug, a suction button, or an air/water supply button.

17. An item for a medical device configured to be stored in an item container for a medical device comprising:
a part of an item container for a medical device including:
a bulging-shape portion configured to bulge in a first direction and to store an item for a medical device in a hollow interior;
a flange provided at a peripheral edge of the bulging-shape portion;
a protruding-shape portion protruding from the flange in the first direction; and
a concave portion provided in contact with the bulging-shape portion,
in which the protruding-shape portion is arranged at a position spaced apart from the bulging-shape portion in a second direction and is provided to extend in an elongated manner in a third direction different from the first direction and the second direction; and
a seal body bonded to the part of the item container for the medical device,
wherein the item for the medical device is stored in the hollow interior of the bulging-shape portion.
